(19) United States
(12) Patent Application Publication
(10) Pub. No.: US 2021/0113357 A1
(43) Pub. Date: Apr. 22, 2021

(54) BRAIDED STRUCTURE, IN PARTICULAR STENT, AND METHOD FOR BRAIDING A BRAIDED STRUCTURE

(71) Applicant: FREISTAAT BAYERN VERTRETEN DURCH HOCHSCHULE HOF,INSTITUT FÜR MATERIAL WISSENS, Hof/Saale (DE)

(72) Inventors: Frank Ficker, Tiefenbrunn (DE); Roxana Miksch, Altenstadt (DE)

(21) Appl. No.: 16/970,460

(22) PCT Filed: Feb. 18, 2019
(86) PCT No.: PCT/EP2019/053991

§ 371 Date: Aug. 17, 2020

(30) Foreign Application Priority Data
Feb. 19, 2018 (DE) 10 2018 103 613.2

(51) Int. Cl. A61F 2/90; D04C 1/06

(52) U.S. Cl. CPC A61F 2/90; D10B 2509/06; D04C 1/06

(57) ABSTRACT

A braided structure and associated method of making include a primary limb braided from a group of filaments and at least two secondary limbs. Each secondary limb is braided from filaments, wherein a totality of the filaments associated with the secondary limbs is the same as the group of filaments of the primary limb. A transitional region is braided from the group of filaments of the primary limb formed between the primary limb and the secondary limbs.

### FIELD OF THE INVENTION

The present invention relates to a braided structure, in particular a stent, having a primary limb and at least two secondary limbs, wherein the primary limb is braided from a group of filaments, in particular threads or wires, and the secondary limbs are braided from filaments such that the totality of the filaments associated with the secondary limbs is the same as the group of filaments of the primary limb. Moreover, the invention relates to a method for braiding the braided structure, in particular a stent, wherein the primary limb is first braided from a group of filaments, in particular threads or wires, and the secondary limbs are then braided, or the method steps are carried out in the reverse order.

### BACKGROUND

In braided structures of the generic type, a primary limb, for example, is first braided from a group of filaments. The carriers for a first secondary limb are then rearranged in the braiding machine, while the carriers for one or multiple further secondary limbs are parked. Thereafter, when the first secondary limb has been braided, the further secondary limbs are braided, one after the other, while the carriers for the particular other secondary limbs are parked. The process continues in this way until all secondary limbs have been braided.

Due to the parking and the necessary manual rearrangement of the carriers, this method is highly complex. In addition, holes arise between the secondary limbs, which inhibit an optimal function of the braided structure as a stent.

The problem addressed by the present invention is therefore that of providing an improved braided structure and an improved method for braiding a braided structure.

### SUMMARY

The problem is solved with the aid of a braided structure and a method for braiding a braided structure having the features described herein. Additional objects and advantages of the invention will be set forth in part in the following description, or may be obvious from the description, or may be learned through practice of the invention.

The invention relates to a braided structure comprising a primary limb and at least two secondary limbs. A braided structure of this type can be, in particular, a stent or a structure for a conduit. Stents have various applications in medicine, for example, for holding blood vessels open or for holding bronchi open. Structures of this type for conduits can be utilized for reinforcement, rehabilitation, or repair of conduits, which are made, for example, of plastic. In addition, various further applications of structures of this type are possible.

The primary limb is braided from a group of filaments. A filament is understood to be a quasi endless, elongate structure. In particular, the filaments can be threads or wires. For medical purposes, it is often advantageous when the wires are made of a shape memory alloy, such as Nitinol.

The secondary limbs are also braided from filaments and, in fact, in such a way that the totality of the filaments associated with the secondary limbs is the same as the group of filaments of the primary limb. In other words, the filaments, from which the primary limb is braided, are subdivided into multiple pieces, wherein a secondary limb is braided from each of these pieces. Filaments from secondary limbs are not required for the manufacture of other secondary limbs outside the transitional region.

According to the invention, a transitional region, which is braided from the group of filaments of the primary limb, is arranged between the primary limb and the secondary limbs. The group of filaments therefore does not transition directly from the primary limb into the secondary limbs, but rather is braided therebetween to form the transitional region. Without a transitional region, i.e., when the primary limb transitions directly into the secondary limbs, a hole arises between the secondary limbs. This hole is partially or completely closed due to the appropriately braided transitional region. A braided structure therefore results, which is considerably improved, in particular for medical purposes.

This improved braided structure advantageously comprises essentially no hole in the braided transitional region. Preferably, there is no hole present either in the gusset region between the secondary limbs, nor at the outer circumference of the transitional region. This means, the maximum size of the individual loops in the transitional region essentially corresponds to the size of the loops of the primary limb and the secondary limb. While a smaller size of the loops in the transitional region can be advantageous in many embodiments, larger loops, for example, having an area of more than the 1.5-times of the loop area in a limb, which represent an impermissible hole, are to be avoided.

One particular advantage of the invention is that the manufacture of the secondary limbs can take place simultaneously. The entire braided structure is therefore manufactured continuously and without a necessary pause for a, in particular, manual reconfiguration of the braiding device. A manual intervention, in particular during the manufacture of the transitional region, is therefore not necessary.

The manufacture of the braided structure can also take place without a core. The installation of different cores for different diameters of the individual loops is therefore not necessary and facilitates the continuous braiding of the structure. Due to the continuous braiding of the structure, no knot-like entanglements of the filaments arise, but rather entanglements in the vertical direction, i.e., in the longitudinal direction of the braid. Purely horizontal entanglements or rearward-directed entanglements do not take place, since the manufacturing progression of the braid takes place in the vertical direction.

Advantageously, the braided structure is continuously braided, in particular on a braider comprising switchable switch-points. Such a braider comprising switchable switch-points is preferably a variation braider, a branch braider, or a 3D braider. Due to the fact that the switch-points are switchable, it is possible to braid the braided structure without manual intervention or with minimal manual intervention. In particular, the transition from the primary limb to the transitional region, the transitional region itself, and the transition from the transitional region to the secondary limbs can be easily braided by switching the switch-points. A removal and installation, rearrangement, and parking of carriers is not necessary, and so the braided structure is relatively easily manufactured.

It is also advantageous when the transitional region comprises a dividing region, which transforms a braided structure of the primary limb into braided structures of the secondary limbs. Certain positions of the carriers in the braiding machine correspond to the individual braided structures, wherein, in general, the positions of the carriers during the braiding of the primary limb do not coincide with positions of the carriers during the braiding of the secondary limbs. The dividing region is braided in order to bring the carriers from one position into the other position. In the case of a clever switch-point position, just a quarter turn of horn gears of the braiding machine can be sufficient.

It is advantageous when the transitional region comprises a crossover region, in which filaments associated with various secondary limbs cross over one another. The crossover region can also simultaneously comprise the dividing region. Due to the crossover of the filaments associated with various secondary limbs, the hole between these limbs is closed. The crossover can take place in various ways. A filament initially associated with one secondary limb can be associated with another secondary limb after the crossover, and vice versa. After the crossover, the filaments can also return to the particular limb with which they were originally associated, however. Moreover, it is possible that not only one filament crosses over in each case, but rather that multiple filaments cross over one another, side by side or one behind the other.

Advantageously, filaments that come from sides of the secondary limb that face away from the particular other secondary limb also cross over one another in the crossover region. Therefore, filaments not only cross over one another from the inner side between the limbs, but also from the outer side. Due to the incorporation of these filaments, the hole between the limbs is closed particularly well.

It is advantageous when two filaments associated, in particular, with various secondary limbs are twisted among one another in the crossover region. A twisting among one another by half a revolution corresponds to a single crossover, wherein the filaments return to the respective limbs with which they were originally associated. In order to better close the hole between the limbs, however, a twisting among one another by a multiple of half a revolution is advantageous. In the case of an uneven multiple of half a revolution, the filaments return to the respective limbs with which they were originally associated; in the case of an even multiple of half a revolution, the filaments switch limbs.

It is advantageous when the transitional region comprises one further dividing region, and so the crossover region is arranged between the dividing region and the further dividing region. In the dividing region, the position of the carriers first transitions from the braided structure for the primary limb to the braided structure for the secondary limbs. In the crossover region, the filaments of the secondary limbs are then crossed over in such a way that the holes between the secondary limbs are closed. The further dividing region now guides the position of the carriers, as it is at the end of the crossover region, into a position that is suitable for braiding the secondary limbs. In this way, a continuous braiding of the individual regions is therefore possible, without the need to manually change carriers.

In one particularly advantageous embodiment of the invention, secondary limbs are arranged at both ends of the primary limb. Therefore, highly complex braided structures can be produced. In particular, it is also possible, however, in the case of a continuous braiding, to produce a quasi endless, braided structure, which comprises a plurality of adjacently arranged primary and secondary limbs, which can be separated, as necessary, into individual braided structures comprising, for example, one primary limb and secondary limbs arranged at only one end of the primary limb.

Preferably, a separation region is provided at the primary limb and/or the secondary limb, which has a loop density and/or loop shape that have/has changed with respect to the rest of the limb, in order to separate the one entire braided structure into multiple individual braided structures. The separation region can be configured in such a way that it advantageously allows for a separation, for example, with the aid of a laser, and, in particular, holds the cut filaments together in a compact manner. These cut and, possibly, protruding filaments can turned inward to form loops, in order to form a blunt end of the braided structure.

Moreover, a method is provided for braiding a braided structure comprising a primary limb and at least two secondary limbs. The braided structure can be, in particular, a stent, wherein this is a branched stent in this case.

The primary limb is first braided from a group of filaments. A filament is understood to be a quasi endless, elongate structure. In particular, the filaments can be threads or wires, wherein it is often advantageous for medical purposes when the wires are made of a shape memory alloy, such as Nitinol.

According to the invention, an essentially hole-free transitional region is then braided from the group of filaments of the primary limb. Without a transitional region, i.e., when the primary limb transitions directly into secondary limbs, a hole arises between the secondary limbs. This hole is partially or completely closed due to the appropriately braided transitional region. The method therefore results in a braided structure, which is considerably improved, in particular for medical purposes.

Finally, the secondary limbs are braided from the group of filaments of the primary limb. The group of filaments is therefore subdivided into multiple pieces, wherein a second limb is braided from each piece.

Of course, the method steps can also be carried out in the reverse order. Therefore, first the secondary limbs, then the transitional region, and finally the primary limb can be braided.

Advantageously, the braided structure is continuously braided, in particular with the aid of a braider comprising switchable switch-points, preferably a variation braider, a branch braider, or a 3D braider. Due to the fact that the switch-points are switchable, it is possible to braid the braided structure without manual intervention or with minimal manual intervention. In particular, the transition from the primary limb to the transitional region, the transitional region itself, and the transition from the transitional region to the secondary limbs can be easily braided by switching the switch-points. A removal and installation, rearrangement, and parking of carriers is not necessary, and so that the braided structure is relatively easily manufactured.

It is also advantageous when the filaments are braided in the transitional region in such a way that they are transformed from a braided structure of the primary limb into braided structures of the secondary limbs, whereby a dividing region is formed. Certain positions of the carriers in the braiding machine correspond to the individual braided structures, wherein, in general, the positions of the carriers during the braiding of the primary limb do not coincide with positions of the carriers during the braiding of the secondary limbs. The dividing region is braided in order to bring the carriers from one position into the other position. In the case of a clever switch-point position, even a quarter turn of horn gears of the braiding machine can be sufficient.

It is advantageous when filaments associated with various secondary limbs are crossed over in the transitional region and therefore form a crossover region. The crossover region can also simultaneously comprise the dividing region. Due to the crossover of the filaments associated with various secondary limbs, the hole between these limbs is closed. The crossover can take place in various ways. A filament initially associated with one secondary limb can be associated with another secondary limb after the crossover, and vice versa. After the crossover, the filaments can also return to the particular limb with which they were originally associated, however. Moreover, it is possible that not only one filament crosses over in each case, but rather that multiple filaments cross over one another, side by side or one behind the other.

Advantageously, filaments that come from sides of the secondary limb that face away from the particular other secondary limb also cross over one another in the crossover region. Therefore, filaments are not only crossed over from the inner side between the limbs, but also from the outer side. Due to the incorporation of these filaments, the hole between the limbs is closed particularly well.

It is advantageous when two filaments associated, in particular, with various secondary limbs are twisted among one another in the crossover region. A twisting among one another by half a revolution corresponds to a single crossover, wherein the filaments return to the respective limbs with which they were originally associated. In order to better close the hole between the limbs, however, a twisting among one another by a multiple of half a revolution is advantageous. In the case of an uneven multiple of half a revolution, the filaments return to the respective limbs with which they were originally associated; in the case of an even multiple of half a revolution, the filaments switch limbs.

If, in one advantageous embodiment of the method, filaments that come from sides of the secondary limb that face away from the particular other secondary limb are also twisted among one another in the crossover region, the holes can be closed in a highly dense manner, since more filaments are available, which are braided in the region of the possible hole.

It is advantageous when one further dividing region is braided in the transitional region, and so first the dividing region, then the crossover region, and finally the further dividing region are braided. In the dividing region, the position of the carriers first transitions from the braided structure for the primary limb to the braided structure for the secondary limbs. In the crossover region, the filaments of the secondary limbs are then crossed over in such a way that the holes between the secondary limbs are closed. The further dividing region now guides the position of the carriers, as it is at the end of the crossover region, into a position that is suitable for braiding the secondary limbs. In this way, a continuous braiding of the individual regions is therefore possible, without the need to manually change carriers.

Preferably, a braided structure, in particular in a separation region, is separated into multiple braided structures. As a result, in one continuous manufacturing method, a strand of a braided structure can be produced, in which primary and secondary limbs alternate. At arbitrary points or also at points predefined by the separation regions, this strand can be separated into individual, smaller braided structures.

The braided structure is designed according to the preceding description and the method for braiding a braided structure is carried out according to the preceding description, wherein the mentioned features can be present individually or in any combination.

### BRIEF DESCRIPTION OF THE DRAWINGS

Further advantages of the invention are described in the following exemplary embodiments. Wherein:

FIG. 1 shows a diagrammatic view of a braided structure known from the related art;

FIG. 2 shows a diagrammatic view of one further braided structure;

FIG. 3 shows a diagrammatic view of one further braided structure;

FIG. 4 shows a diagrammatic view of one further braided structure;

FIG. 5 shows a diagrammatic view of a crossover region;

FIG. 6 shows a diagrammatic view of one further crossover region;

FIG. 7 shows a diagrammatic view of one further crossover region;

FIG. 8 shows a diagrammatic view of one further crossover region;

FIG. 9 shows a diagrammatic view of one further crossover region;

FIGS. 10a-10c show braiding program steps for a dividing region;

FIGS. 11a-11e show braiding program steps for a transitional region; and

FIG. 12 shows a diagrammatic view of one further braided structure similar to FIG. 2 comprising a separation region.

### DETAILED DESCRIPTION

Reference will now be made to embodiments of the invention, one or more examples of which are shown in the drawings. Each embodiment is provided by way of explanation of the invention, and not as a limitation of the invention. For example features illustrated or described as part of one embodiment can be combined with another embodiment to yield still another embodiment. It is intended that the present invention include these and other modifications and variations to the embodiments described herein.

In the following description of alternative exemplary embodiments, the same reference numbers are utilized for features that are identical and/or at least comparable to other exemplary embodiments in terms of their design and/or mode of operation. Provided the features are not described in detail again, their design and/or mode of operation correspond/corresponds to the design and mode of operation of the above-described features.

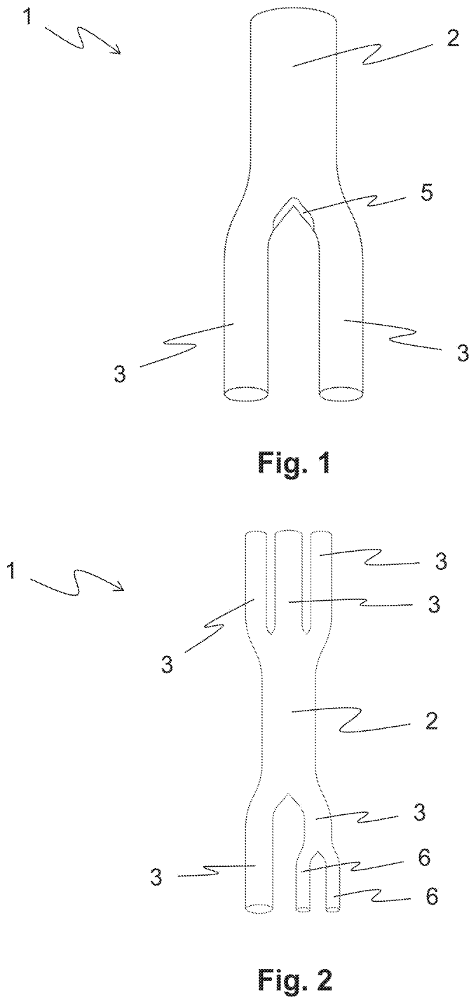

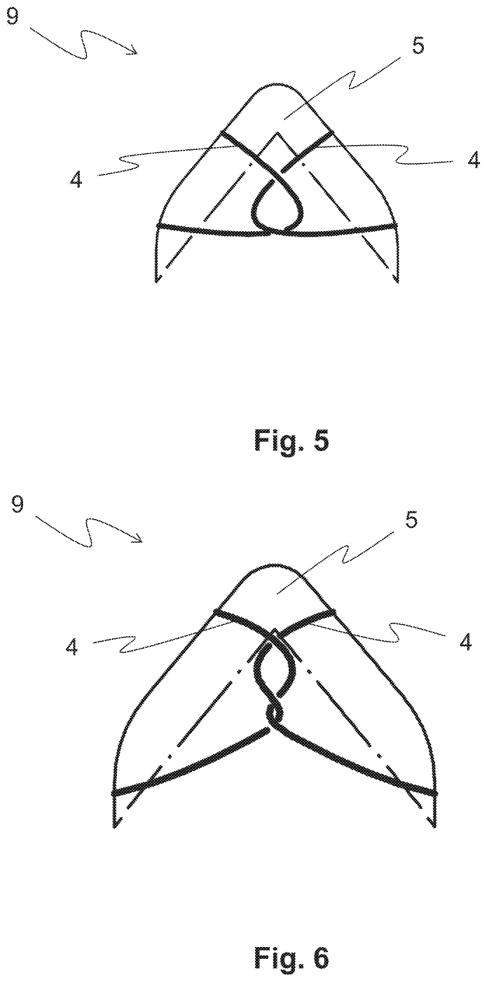

FIG. 1 shows a braided structure 1, which is known from the related art. The braided structure 1 comprises a primary limb 2 and two secondary limbs 3. The individual filaments 4 (FIG. 5), from which the braided structure 1 is braided, are not represented in this figure. The totality of the filaments 4 from which the secondary limbs 3 are braided is the same as the group of filaments 4 from which the primary limb 2 is braided. Possibilities for filaments 4 are highly diverse, quasi endless, elongate structures, in particular threads and wires.

During the transition from the primary limb 2 to the secondary limbs 3, a hole 5 arises between the secondary limbs 3. This hole 5 is disadvantageous for many possible applications of the braided structure 1. One possible application of this type is the utilization of the braided structure 1 as a stent. The stent holds vessels or hollow organs open, in particular blood vessels or bronchi. When the braided structure 1 is utilized as a stent, the filaments 4, advantageously wires, are made of a shape memory alloy, such as Nitinol. The stent can be, for example, bound, in order to be introduced into the vessel or the hollow organ and, after the binding is released, the stent is deployed to resume its original shape. A hole 5 between the secondary limbs 3 of a branched stent means that neither a support of the vessels or hollow organs occurs in this region, nor can drugs be effectively applied onto the surface of the stent in this region.

FIG. 2 shows a diagrammatic view of a braided structure 1 according to the invention, in which the holes 5 between the secondary limbs 3 are closed. In this complex braided structure 1, the primary limb 2 branches in one direction into three secondary limbs 3. In the other direction, the primary limb 2 first branches into two secondary limbs 3, wherein one of the secondary limbs 3 branches into two further limbs 6. With respect to the branching and the braid pattern resulting within the scope of this branching, the secondary limb 3 functions in this case as a primary limb and the further limbs 6 function as secondary limbs. Of course, many further branching variants are possible within the scope of the claims, from a simple branched structure, as shown in FIG. 1, up to complex structures comprising a plurality of branchings.

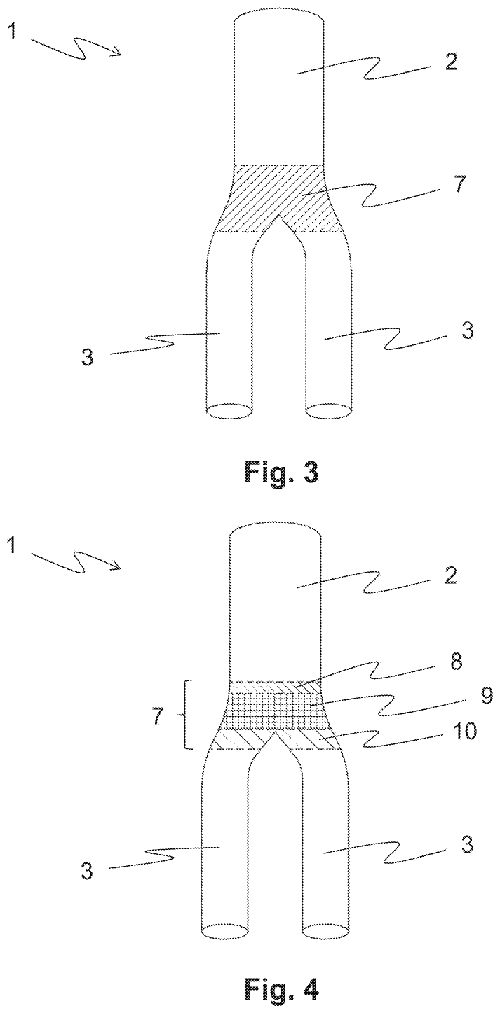

In the braided structure 1 shown in FIG. 3, a transitional region 7 is provided between the primary limb 2 and the secondary limbs 3. This transitional region 7 is braided from the same group of filaments 4 from which the primary limb 2 and the secondary limbs 3 have been braided.

As is also shown in the other exemplary embodiments, the two secondary limbs 3 extend essentially in parallel to the primary limb 2. The secondary limbs 3 therefore do not spread apart from one another, but rather divide the primary limb 2 and extend further essentially in the same direction as the primary limb 2. Even if it were possible, in principle, to allow the secondary limbs 3 to spread apart from the primary limb 2, it is advantageous when the secondary limbs 3 extend essentially in parallel to the primary limb 2, in particular within the scope of a continuous production of the structure 1 while simultaneously manufacturing all secondary limbs 3. If it should be necessary, for a special application, for the secondary limbs 3 to spread apart, this can be brought about, for example, with the aid of a thermal aftertreatment.

FIG. 4 shows one possible embodiment of the transitional region 7. The primary limb 2 is followed by a dividing region 8. This dividing region 8 transforms a braided structure of the primary limb 2 into braided structures of the secondary limbs 3. The dividing region 8 is followed by a crossover region 9. In the crossover region 9, filaments 4 associated with the first secondary limb 3 and the second secondary limb 3 cross over one another. As a result, the hole 5, which would otherwise form between the secondary limbs 3, is closed. The crossover region 9 is followed by one further dividing region 10, which transforms a braided structure prevailing downstream from the crossover region 9 into the braided structures of the secondary limbs 3. This further dividing region 10 is followed by the two secondary limbs 3.

Of course, more than two secondary limbs 3 are also conceivable. In that case, crossovers can take place in the crossover region 9 in such a way that filaments 4 from each of the secondary limbs 3 cross over one another. Moreover, it is possible that only one dividing region 8 is provided and/or that the dividing region 8 is identical to the crossover region 9, i.e., the braided structure of the primary limb 2 is also simultaneously transformed into the braided structures of the secondary limbs 3 within the scope of the crossover of the filaments 4.

FIGS. 5 through 8 show the crossover region 9 at the transition from the primary limb 2 to the secondary limbs 3, where the hole 5 would be located without the crossovers.

FIG. 5 shows a single crossover of two filaments 4, which corresponds to half a revolution of a twisting of the filaments 4. After the crossover, the filaments 4 return to the secondary limbs 3 from which they came. This crossover results in a simple closure of the hole 5.

FIG. 6 shows a twisting of the filaments 4 by a full revolution. The filaments 4 switch from one secondary limb 3 to the other. Due to the longer twisting of the filaments 4, the hole 5 is closed in a better way than is the case for the single crossover.

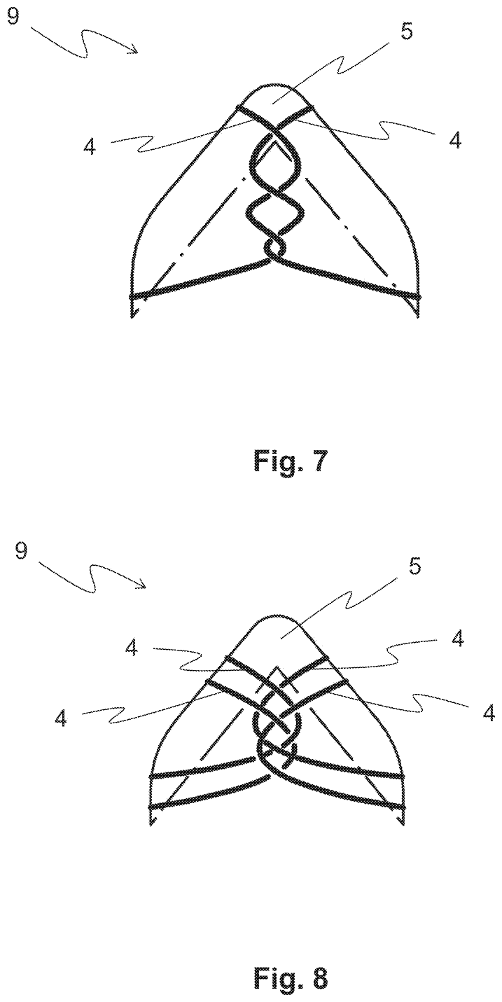

A twisting of the filaments 4 by three half revolutions, as shown in FIG. 7, results in the filaments 4 returning to the secondary limbs 3 from which they came. The hole 5 is closed due to the relatively long twisting of the filaments 4.

FIG. 8 shows one further exemplary embodiment of the crossover region 9, in which two filaments 4 of each of the secondary limbs 3 are involved in the crossover. The crossover is a single crossover in this case. It is also possible, however, to carry out twistings about one full revolution or more with two filaments 4. It is also possible that more than two filaments 4 per secondary limb 3 cross over one another.

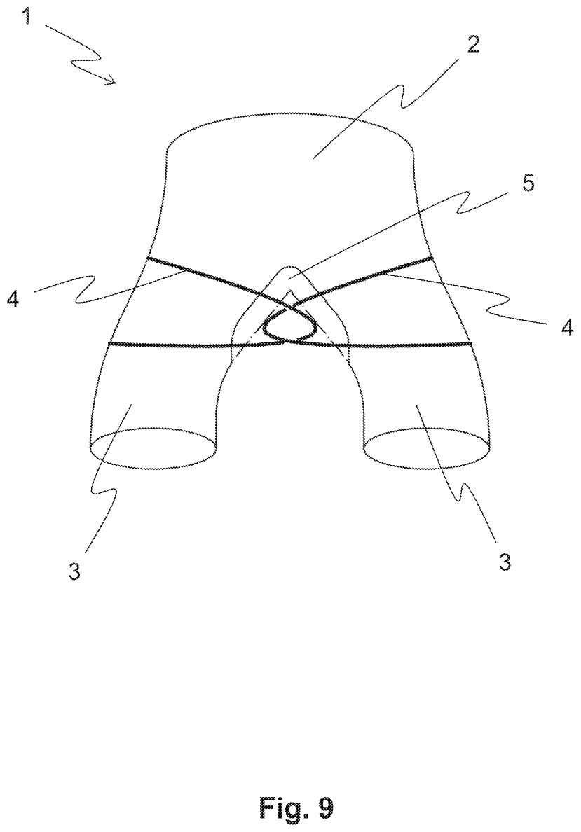

In the exemplary embodiment shown in FIG. 9, filaments 4, which come from sides of the secondary limbs 3 facing away from the particular other secondary limb 3, cross over one another in the crossover region 9; in other words, filaments 4 cross over one another from the outer sides of the secondary limbs 3. Due to this crossover, the hole 5 is closed and the secondary limbs 3 are additionally held together.

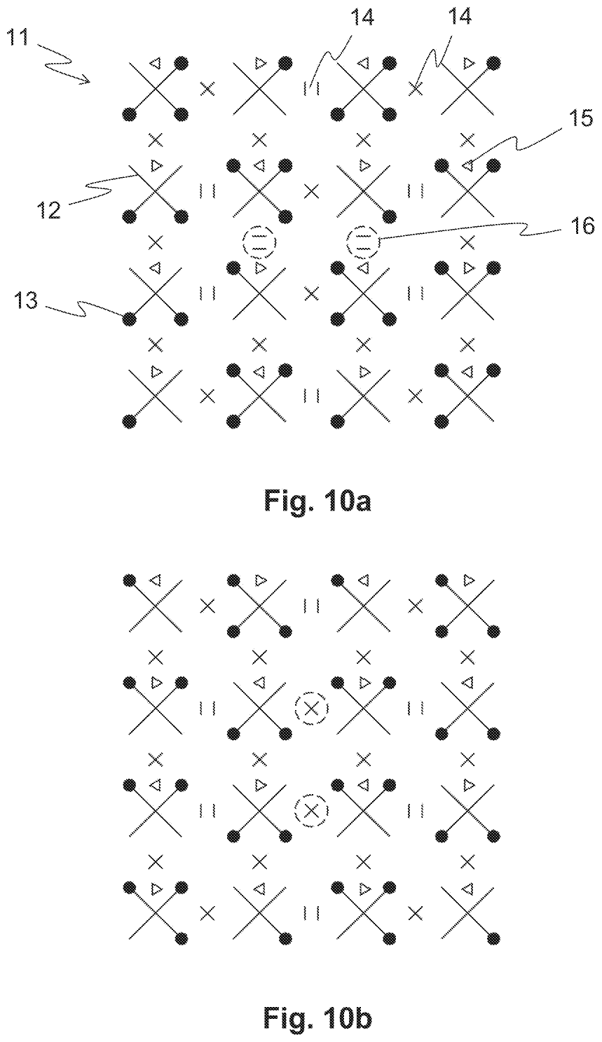

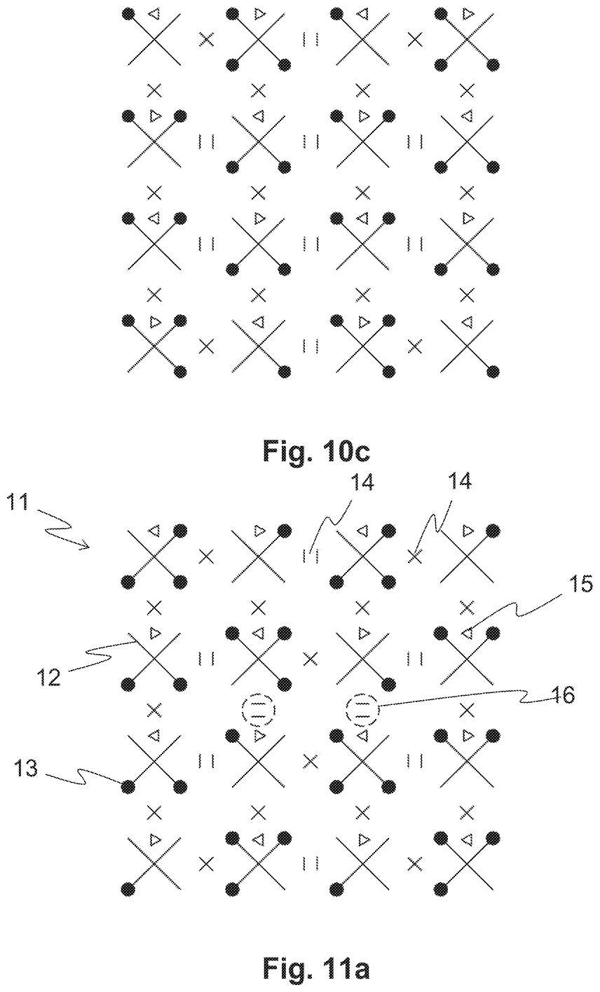

FIGS. 10a through 10c show braiding program steps for a dividing region 8 on a variation braider 11 comprising 4×4 horn gears 12. In FIG. 10a, the configuration comprising carriers 13 and the position of the switch-points 14 for the braiding of the primary limb 2 are represented. With respect to the switch-points 14, an “x” means that a crossover of the carriers 13 takes place, and an “∥” or “=” means that no crossover of the carriers 13 takes place. The direction of rotation 15 of the horn gears 12 is indicated with the aid of a small triangle.

The switch-points 16 to be switched, which are indicated by a dashed border, are now switched for the braiding of the dividing region 8. The horn gears 12 are turned further by 90°. The configuration including carriers 13 and the position of the switch-points 14 from FIG. 10b result. In order to end the dividing region 8, the switch-points 16 to be switched in FIG. 10b are switched. The configuration including carriers 13 and the position of the switch-points 14 from FIG. 10c result; thereafter, two secondary limbs 3 are braided.

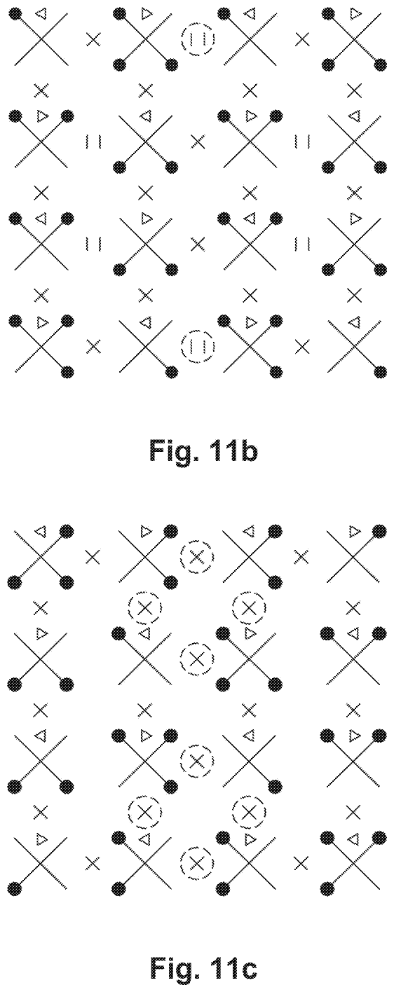

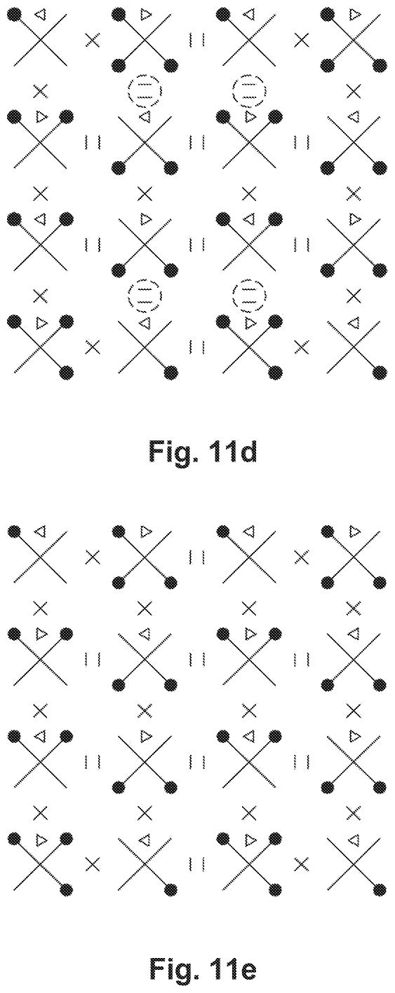

FIGS. 11a through 11e show braiding program steps for a transitional region 7, which combines a dividing region 8 and a crossover region 9. The starting point of FIG. 11a, as in FIG. 10a, is the configuration including carriers 13 and the position of the switch-points 14 for the braiding of the primary limb 2. After switching the switch-points 16 to be switched and turning the horn gears 12 by 90°, the configuration including carriers 13 and the position of the switch-points 14 from FIG. 11b results. The switch-points 16 to be switched are now positioned again and the horn gears 12 are turned by 90°, in order to obtain the configuration including carriers 13 and the position of the switch-points from FIG. 11c. Once more, the switch-points 16 to be switched are switched and the horn gears 12 are turned by 90°, in order to obtain the configuration including carriers 13 and the position of the switch-points from FIG. 11d. After the switching of the switch-points 16 to be switched, the configuration including carriers 13 and the position of the switch-points from FIG. 11e is obtained; thereafter, two secondary limbs are braided, as in FIG. 10c. In four short steps, the dividing region 8 and the crossover region 9 are therefore simultaneously braided.

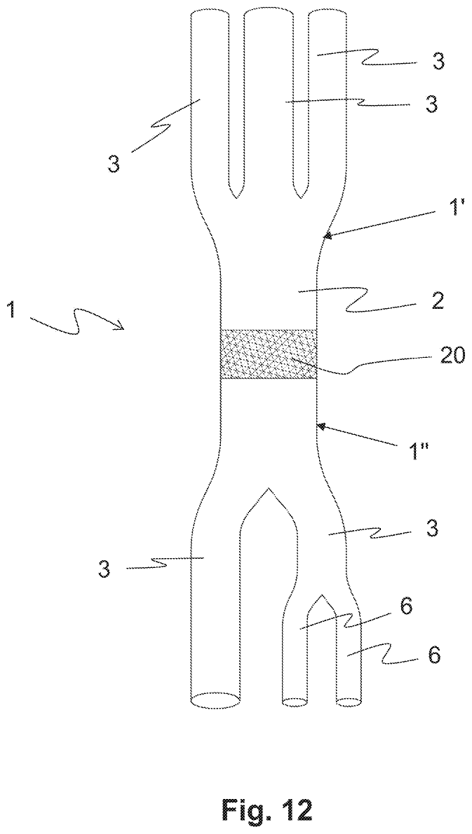

FIG. 12 shows a diagrammatic view of one further braided structure similar to FIG. 2 comprising a separation region 20 at the primary limb 2. The complex braided structure 1 shown here is preferably cut apart in the separation region 20. The cutting can take place, for example, with the aid of a laser cut. As a result, two braided structures 1′ and 1″ are obtained. Alternatively or additionally, the separation region 20 can be arranged at one or multiple secondary limb(s) 3 or 6 (not shown).

A changed loop structure of the filaments 4 is indicated in the separation region 20. During the cutting of the structure 1 in the separation region 20, it can be brought about, as a result, that the filaments 4 are advantageously, for example, more compactly, arranged at the resultant end of the structure 1′ and 1″ and therefore form an advantageous termination of the structure 1′ and 1″.

Of course, multiple structures 1′ and 1″ can be adjacently arranged and, therefore, multiple separation regions 20 can also be provided (not shown). In principle, a separation of a complex structure of this type can also be possible without separation regions 20 being provided. The loose ends of the filaments 4 can appropriately protrude or be further processed, depending on the application, and so a blunt end of the structure 1′ or 1″ is produced, for example, by forming a loop.

The present invention is not limited to the represented and described exemplary embodiments. Modifications within the scope of the claims are also possible, as is any combination of the features, even if they are represented and described in different exemplary embodiments.

1-19. (canceled)

20. A braided structure, comprising:
a primary limb braided from a group of filaments;
at least two secondary limbs, each secondary limb braided from filaments, wherein a totality of the filaments associated with the secondary limbs is the same as the group of filaments of the primary limb; and
a transitional region braided from the group of filaments of the primary limb formed between the primary limb and the secondary limbs.

21. The braided structure as in claim 20, wherein the transitional region is essentially free from holes.

22. The braided structure as in claim 20, wherein the braided structure is a continuously braided structure.

23. The braided structure as in claim 20, wherein the transitional region further comprises a first dividing region that transforms a braided structure of the primary limb into braided structures of the secondary limbs.

24. The braided structure as in claim 23, wherein the transitional region further comprises a crossover region in which the filaments associated with the secondary limbs cross over one another.

25. The braided structure as in claim 24, wherein the filaments that come from sides of the secondary limbs that face away from each other also cross over in the crossover region.

26. The braided structure as in claim 24, wherein two filaments associated with the secondary limbs are twisted together in the crossover region.

27. The braided structure as in claim 23, wherein the transitional region comprises a second dividing region such that the crossover region is arranged between the first and the second dividing regions.

28. The braided structure as in claim 20, wherein secondary limbs are arranged at both ends of the primary limb.

29. The braided structure as in claim 20, further comprising a separation region in the primary limb or in one or more of the secondary limbs, the separation region comprising one or both of a loop density or loop shape that is different with respect to primary limb or secondary limb in which it is located to facilitate separation of the braided structure at the separation region into multiple individual braided structures.

30. A method for braiding a braided structure having a primary limb and at least two secondary limbs, comprising:
braiding the primary limb from a group of filaments;
at a transitional region between the primary limb and the secondary limbs, braiding a hole-free transitional region from the group of filaments of the primary limb; and
braiding the secondary limbs, wherein a totality of the filaments associated with the secondary limbs is the same as the group of filaments of the primary limb.

31. The method as in claim 30, wherein the braided structure is continuously braided with a braider having switchable switch-points.

32. The method as in claim 30, the filaments are braided in the transitional region so as to form a dividing region and transform from a braided structure of the primary limb into braided structures of the secondary limbs.

33. The method as in claim 30, wherein the filaments associated with the secondary limbs are crossed over in a crossover region of the transitional region.

34. The method as in claim 33, wherein the filaments that come from sides of the secondary limbs that face away from each other are crossed over in the crossover region.

35. The method as in claim 33, wherein two filaments associated with the secondary limbs are twisted together in the crossover region.

36. The method as in claim 33, wherein the transitional region is braided to have a first dividing region that transforms a braided structure of the primary limb into braided structures of the secondary limbs.

37. The method as in claim 36, wherein the transitional region is braided to have a second dividing region such that the crossover region is arranged between the first and the second dividing regions.

38. The method as in claim 30, further comprising separating the braided structure at a separation zone in multiple braided structures each having a primary limb and secondary limbs.
